(19) United States
(12) Patent Application Publication
(10) Pub. No.: US 2011/0171167 A1
(43) Pub. Date: Jul. 14, 2011

(54) Isocyanate-Based Compositions and Their Use

(75) Inventor: Scott Herring, Longmont, CO (US)

(73) Assignee: TYCO HEALTHCARE GROUP LP, North Haven, CT (US)

(21) Appl. No.: 11/630,232

(22) PCT Filed: Jun. 30, 2005
(86) PCT No.: PCT/US05/23147

§ 371 Date: Nov. 7, 2007

Related U.S. Application Data
(60) Provisional application No. 60/584,341, filed on Jun. 30, 2004.

(51) Int. Cl. A61K 31/785

(52) U.S. Cl. 424/78.27

(57) ABSTRACT

The present disclosure describes a method and a kit for bulking, augmenting or occluding a tissue comprising administering a sufficient amount of a composition containing a biocompatible, non-bioabsorbable isocyanate-based material onto or into tissue.

The present disclosure relates to methods of dermal augmentation, lumen filling, tissue bulking, and the like using injectable compositions.

The terms “present composition” or “composition in accordance with the present disclosure” or “compositions of the present disclosure” and the like as used in the following disclosure refer to a composition that includes an isocyanate-based material. Suitable isocyanate-based materials include, but are not limited to one or more of: i) the biocompatible tissue-bonding adhesive compositions disclosed in published international application WO 03/049637 A2; ii) the in-situ polymerizing medical compositions disclosed in published U.S. application US 2004/0068078 A1; and the tissue augmentation compounds disclosed in U.S. Pat. No. 6,702,731. The entire disclosure of each of these publications is incorporated herein in its entirety by this reference. It should be understood that the present compositions may include any other biocompatible material in combination with the isocyanate-based material. The additional biocompatible components can be bioabsorbable or non-bioabsorbable.

In accordance with one embodiment of this disclosure, the present compositions are used to increase bulk in order to increase the competency of sphincter muscles located throughout the body. This involves injection or other administration of the present composition directly into the sphincter muscles. See U.S. Pat. No. 5,490,984, where this approach using collagen has been shown to alleviate anorectal and/or urinary incontinence. This increase in muscle bulk counteracts the stretched condition of a muscle, tightening it, and thereby aiding in the treatment of an individual having incontinence problems due to a weakened or stretched muscle of the urethra. Thus, the subject methods can be used to treat incontinence due to incompetent sphincter muscles along the GI and urinary tracts. Treatment involves the injection of the present compositions directly into the sphincter muscles. Also, injecting such a composition into vocal chords bulk up this area, leading to a change in voice characteristics.

In one embodiment, the present disclosure relates to methods for treating Gastroesophageal Reflux Disease (“GERD”). Although gastroesophageal reflux is a normal physiological phenomenon, in some cases it is a pathophysiological situation that can result in a variety of symptoms which may become severe in extreme cases. Gastro-Esophageal Reflux Disease (“GERD”) describes a backflow of acidic and enzymatic liquid from the stomach to the esophagus. It causes burning sensations behind the sternum that may be accompanied by regurgitation of gastric acid into the mouth or even the lung. Complications of GERD which define the severity of the disease include esophageal tissue erosion, and esophageal ulcer wherein normal epithelium is replaced by a pathological tissue.

In normal patients, after a meal the lower esophageal sphincter remains closed, but in patients with GERD, it relaxes and allows some acidic material to reflux into the esophageal tube as a result of stomach contractions. Actually GERD can be attributed primarily to transient relaxation of the lower esophageal sphincter. In other cases, GERD can be attributed to decreased resting tone of the lower esophageal sphincter or to congenital small dimension of the sphincter itself. Other causes also exist which contribute to varying degrees to the existence and severity of this disease.

Prior to the present disclosure, in an attempt to increase the function of the sphincter, bulking methods using bovine collagen and Teflon paste have been used in patients. Both methods have been unsuccessful, however, as these materials migrate over time from the initial site of implantation.

For the treatment of GERD in accordance with the present methods, the present compositions can be introduced via the esophagus, either by endoscopic delivery or by laparoscopic technique, and are injected into the walls of the sphincter where the esophagus meets the stomach, i.e., the lower esophageal sphincter. This decreases the internal lumen of the sphincter muscle thus permitting easier contraction of the muscle with reduced regurgitation of the gastric fluids into the esophagus. In addition, this treatment reduces the inflammation of the lower esophagus. The present compositions may also be loaded with X-ray opaque dye or other imaging agents for subsequent X-ray visualization.

In another embodiment, the present compositions can be injected into the sphincter at the junction of the esophagus and stomach in order to treat GERD may also include an amount of a drug used to treat GERD, such as H₂ histamine antagonists including cimetidine, ranitidine, famotidine and nizatidine; inhibitors of H+,K+-ATPase including omeprazole and lansoprazole; antacids including e.g., Al(OH)₃, Mg(OH)₂, and CaCO₃. As with the treatment of urinary incontinence, urinary reflux disease, and skin wrinkles, the compositions may also be used with anti-inflammatory agents, angiogenesis inhibitors, radioactive elements, and antimitotic agents.

Other therapeutic agents to be used in combination with the compositions in accordance with the present disclosure include those for the treatment of skin disorders, GERD, urinary incontinence and urinary reflux disease as reported in Goodman & Gilman's The Pharmacological Basis of Therapeutics, 9th Ed., McGraw-Hill (1996) and The Physicians's Desk Reference® 2000.

### Urinary Incontinence and Urinary Reflux Disease

Urinary incontinence is a prevalent problem that affects people of all ages and levels of physical health, both in the community at large and in healthcare settings. Incontinence can be attributed to genuine urinary stress (urethra hypermobility), to intrinsic sphincter deficiency (“ISD”), or both. It is especially prevalent in women, and to a lesser extent incontinence is present in children (in particular, ISD), and in men following radical prostatectomy.

In accordance with urinary reflux disease, or “vesicoureteral reflux” in its medical term, simply means that urine goes backwards in the ureters during urination. The disease often occurs in young children. The ureter is the tube which connects the kidneys with the bladder. Urine is supposed to go in one direction: from the kidneys to the bladder. When urine goes up from the bladder to the kidneys, it can result in health problems for the person.

Urinary reflux can lead to kidney damage. Refluxing urine can carry bacteria to the kidney, where it can cause kidney infection. Children with reflux of urine are much more likely to have kidney infection than children who do not have reflux. The combination of reflux and infection can lead to areas of permanent kidney damage or “renal scarring.” This scarring is detected by doing an X-ray called an intravenous pyelogram (IVP), or preferably, a renal scan. If it is extensive enough, the scarring can lead to loss of function of one or both kidneys.

The key to preventing renal scarring is preventing kidney infections. This is currently being carried out in two ways. In most cases, long term prophylactic antibiotics are given. The other method of preventing urinary tract infections is surgical correction of the reflux. Both methods, however, have drawbacks. Long term use of antibiotics may cause unpredictable side effects and surgical procedures involve unnecessary risks.

Even though many urinary reflux disease will go away on its own in children, some cases often lead to severe kidney and urinary tract infections and even total kidney failure. There is a need, therefore, for a safe, effective, less intrusive, and long lasting method of treating urinary reflux disease.

A recent approach for the treatment of urinary incontinence associated with ISD is to subject the patient to periurethral endoscopic collagen injections. This augments the bladder muscle in an effort to reduce the likelihood of bladder leakage or stress incontinence. Despite a limited success rate, transurethral collagen injection therapy remains an acceptable treatment for intrinsic sphincter deficiency, due to the lack other suitable alternatives.

For the treatment of urinary incontinence and urinary reflux disease, the compositions of the present disclosure are injectable through needles (e.g., of about 18 gauge to about 26 gauge, preferably, 22 to 24 gauge) and are not capable of being eliminated through the lymphatic system. The present compositions are introduced via the urethra and injected into the walls of the bladder sphincter, decreasing the internal lumen of the sphincter muscle thus permitting easier contraction of the muscle with reduced likelihood of incontinence. The composition may also be loaded with X-ray opaque dye, or other imaging agents for subsequent X-ray visualization.

In another embodiment, the present compositions are injected into the bladder sphincter in order to treat urinary incontinence or urinary reflux disease and also include an amount of a drug used to treat urinary incontinence or urinary reflux disease, such as antidiuretics, anticholinergics, oxybutynin and vasopressins.

Injected compositions can generate some transient adverse reactions such as local inflammation, therefore the present compositions can contain or be injected with anti-inflammatory drugs, such as salicylic acid derivatives including aspirin; para-aminophenol derivatives including acetaminophen; non-steroidal anti-inflammatory agents including indomethacin, sulindac, etodolac, tolmetin, diclodfenac, ketorolac, ibuprofen, naproxen, flurbiprofen, ketoprofen, fenoprofen, oxaprozin; anthranilic acids including mefenamic acid, meclofenamic acid; enolic acids such as piroxicam, tenoxicam, phenylbutazone, oxyphenthatrarone; nabumetone; Vioxx® and Celebrex™. These anti-inflammatories are preferably released slowly over a short period of time (a few days). The compositions may also be used to release other specific drugs which can be incorporated within the composition before injection into the patient. The drug would be released locally at the site of implantation over a short period of time to improve the overall treatment.

Incorporation of active molecules, such as drugs, into the compositions of the present disclosure can be accomplished by mixing the compositions with solutions of said active molecules or drugs in an aqueous or hydro-organic solution.

### Tissue Deficiencies

In another embodiment, the present disclosure relates to methods of treating skin deficiencies. Damage to the skin due to aging, environmental exposure to the sun and other elements, weight loss, child bearing, disease such as acne and cancer, and surgery often results in skin contour deficiencies and other skin anomalies. The use of injectable material (e.g., collagen, silicone) for soft tissue augmentation is a method often used in order to correct contour deficiencies and other anomalies of the skin. The advantage of using hypodermic needles as a delivery device for dermal augmentation reflects the advantages of using hypodermic needles in general: easy, precise and, usually, non-invasive deliveries. Yet, the requirement for such use is also quite strict: the material to be delivered must be deliverable through the needles, which means the material must be able to easily pass through the hollow centers of the needles.

Solid microparticles have also been used for the correction of skin deficiencies and for tissue bulking. For example, carbon particles, silicone particles, TEFLON paste, collagen beads and polymethylmethacrylate spheres, have been used with disappointing results due to, inter alia, adverse tissue reactions, biological degradation and migration from the initial implantation location.

The dermal augmentation method of the present disclosure comprises administering a composition in accordance with this disclosure to a mammal in need of such treatment. The composition is injectable through needles (e.g., of about 30 guage or smaller) and the compositions are not capable of being digested or eliminated through macrophages or other elements of the immune system. The present compositions are preferably injected into the mammal's subcutaneous layer. The compositions may also include one or more anti-inflammatory agents.

Suitable for treatment using the dermal augmentation method of the present disclosure are skin contour deficiencies caused by various conditions including, but not limited to, aging, environmental exposure, weight loss, child bearing, surgery, disease such as acne and cancer, or combinations thereof. The dermal augmentation method of the present disclosure is particularly suitable for skin contour deficiencies such as frown lines, worry lines, wrinkles, crow's feet, facial scars, marionette lines, stretch marks, surgical scars, wounds, and cuts and bites due to injury or accidents.

The present disclosure additionally provides methods of dermal augmentation and treatment of skin deficiency. Specifically, the disclosure provides a method of causing dermal augmentation in a mammal by administering a composition in accordance with this disclosure to the mammal. The composition is injectable through a needle and the compositions are not capable of being digested or eliminated by macrophage or other elements of the mammal's immune system. According to the present disclosure, a preferred method-of administration is injecting the composition into an area of the subject mammal that is in need of dermal augmentation. A more preferred method of administration is injecting the composition into the subcutaneous layer of the subject mammal.

The dermal augmentation method of the present disclosure is especially suitable for the treatment of skin contour deficiencies, which are often caused by aging, environmental exposure, weight loss, child bearing, injury, surgery, in addition to diseases such as acne and cancer. Suitable for the treatment by the present disclosure's method are contour deficiencies such as frown lines, worry lines, wrinkles, crow's feet, marionette lines, stretch marks, and internal or external scars resulted from injury, wound, bite, surgery, or accident. The disclosure also encompasses the use of the injectable compositions to treat skin deficiencies caused by diseases such as acne and cancer.

In yet another embodiment, the present compositions are used for cosmetic enhancement. By injecting the present compositions, enhancement of the size and/or appearance of a patient's cheeks, lips, breast or penis can be achieved.

The present disclosure also provides methods of causing tissue bulking or dermal augmentation by injecting the injectable composition not directly into the body, but extracorporeally into organs, components of organs, or tissues prior to their inclusion into the body, organs, or components of organs.

The injection of the present methods can be preferably carried out by any type of sterile syringes with needles (e.g., of about 18 to 26 gauge). The size of the syringe and the length of the needle used will dependent on the particular injection based on factors such as the specific disease or disorders being treated, the location and depth of the injection, and the volume and specific composition of the injectable suspension being used. A skilled practitioner will be able to make the selection of syringe and needle based on experience and the teaching of the present disclosure.

### Pyloric Bulking

Pyloric obstructions occur in some infants and occasionally in adults wherein ingested food cannot pass through the pylorus lumen in sufficient quantity to provide adequate nutrition. The stomach fills and its contents are then regurgitated. Infants suffer malnutrition and failure to thrive unless surgical procedures are undertaken to correct the obstruction. Thus, the present methods can be employed in treating obese adults so that the induced partial pyloric obstruction or small intestine obstruction prolongs emptying of the stomach or small intestine to induce the patient to refrain from eating frequently or eating too much.

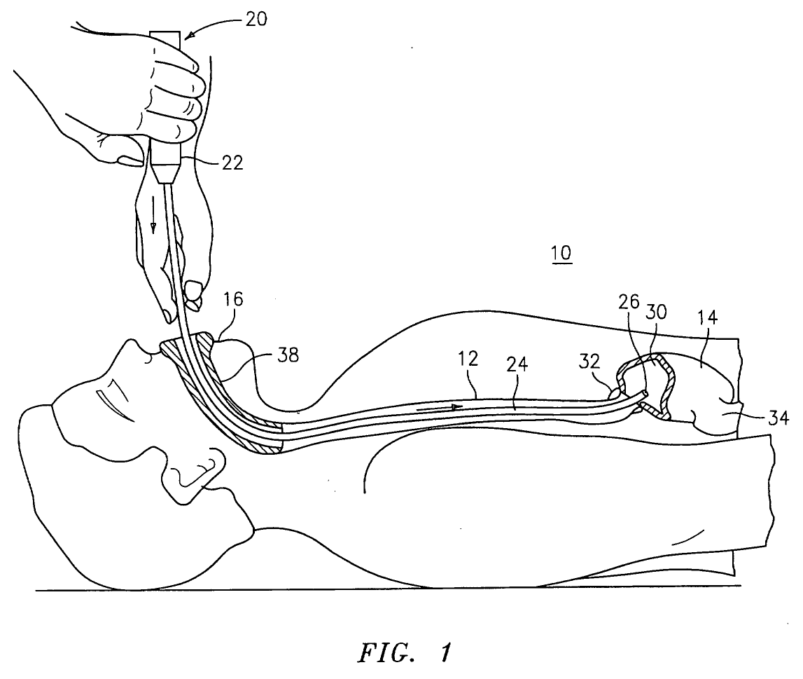

FIG. 1 is a schematic view of obtaining access into the stomach 14 of a patient 10 employing a delivery instrument 20 to enable the implantation of a mass of the present compositions as a bulking agent within the wall of the pylorus or the small intestine as described further below. The delivery instrument 20 comprises a handle 22 coupled to the proximal end of an elongated instrument body 24 extending to an instrument body distal end 26 and enclosing at least one delivery lumen. The delivery instrument 20 encloses at least one instrument lumen distal end opening at instrument body distal end 26.

The delivery instrument 20 can take the form of the instruments described in U.S. Pat. Nos. 6,251,063, 6,251,064, and 6,358,197 (the disclosures of which are incorporated herein in their entirety by this reference) that are employed to inject a mass or masses of bulking agents within the wall of the esophagus in the region of the lower esophageal sphincter (LES) or into the rectal wall in the region of the anal sphincter that solidify in situ. As used herein the terms “mass” and “masses of bulk agents” and the like refer to a three dimensional volume formed at least in part of one or more compositions in accordance with the present disclosure. Alternatively, the delivery instrument 20 can take the form of the instruments set forth in U.S. Pat. Nos. 6,098,629, 6,338,345, and 6,401,718, (the disclosures of which are incorporated herein in their entirety by this reference) that are employed to insert pre-formed prosthetic bulking devices below the mucosa in the region of the LES. The implantation of the mass(es) of bulking agent(s) within the mucosa in the region of the LES is intended to treat patients suffering from gastroesophageal reflux disease (GERD). The mass(es) of bulking agents add bulk to the LES to elevate the LES closing pressure or function as valve mechanisms. The delivery of bulking agents through endoscopes or other instruments into periurethal tissue at the site of a defect to correct urinary incontinence or vesicoureteteral reflux is also disclosed in U.S. Pat. Nos. 5,667,778, 5,755,658, and 5,785,642, (the disclosures of which are incorporated herein in their entirety by this reference). Preferably, the delivery instrument 20 incorporates the imaging features of an endoscope or gastroscope, the delivery lumen(s) for delivering the mass(es) of bulking agent(s), and a retractable cutting or penetrating tip or other mechanism that is selectively actuable to perforate the mucosa to enable advancement of the mass(es) of bulking agent(s) therethrough.

In accordance with the one embodiment of the present disclosure, the instrument body 24 is inserted through a curved mouth and throat guard 38 inserted into the patient's mouth 16, and the instrument body distal end 26 is advanced through the esophagus 12 and LES 32 and into the stomach cavity 30. The instrument body distal end 26 is advanced either to the pylorus 34 or further through the duodenum and to an implantation site of the small intestine. The instrument distal end 26 is directed to the site of implantation in the intestinal wall or the wall of the pylorus 34, and the mass(es) of bulking agent are implanted in one of the ways described further below.

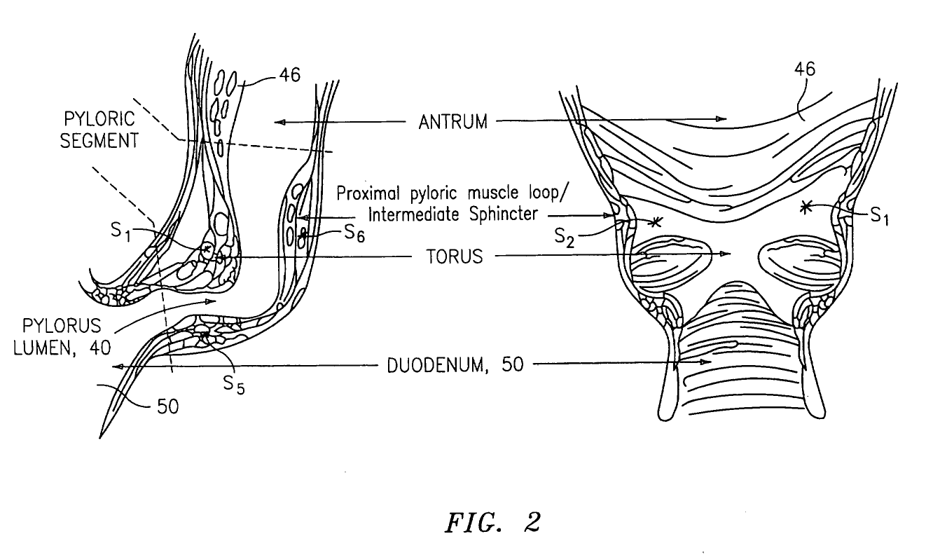

FIG. 2 depicts the pylorus 34 between the stomach 14 and the duodenum 50 in greater detail. In the illustrated embodiments, the mass(es) of bulking agent(s) can be implanted within the submucosa 44 between the mucosal surface or mucosa 46 and the pyloric sphincter 48. Within the stomach proper, the submucosa 44 is a fibrous layer of tissue separating the mucosa 46 and the muscularis externa which itself comprises oblique, circular and longitudinal muscle layers.

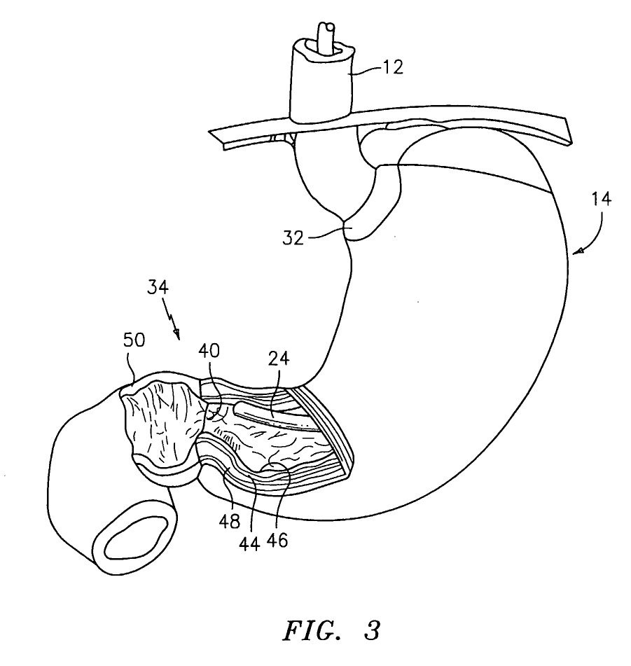

FIG. 3 depicts the pylorus 34 in longitudinal and mucosal section views and showing where the mass(es) of bulking agent can be implanted in the pylorus wall 42 in relation to the labeled parts of the pylorus 34. A submucosal space, that is a potential space, can be created between the mucosa 46 and the pyloric sphincter 48 by the separation of mucosa 46 from the pyloric sphincter 48. The submucosa 44 is a springy tissue that can be separated apart by a blunt instrument or cut using mechanical cutting techniques or cautery tools in order to create a submucosal space or site for implantation of a mass of bulking agent or bulking device. It is expected that bulking agents composed at least in part of one or more the present compositions can be directly injected into the submucosa 44 to displace submucosal tissue and solidify in situ to form a mass or implant of non-biodegradable bulking agent. Alternatively, a submucosal space or site for implantation of a mass of bulking agent or bulking device can be created intramuscularly by distension and separation of muscle fibers of the pyloric sphincter 48.

The pyloric sphincter 48 comprises an intermediate sphincter loop and a distal sphincter loop joined in the shape or a torus. The mass(es) of bulking agent can be implanted adjacent the intermediate sphincter loop at sites S₁ and S₂ or in various ones of the sites S₁ through S₇ to efficaciously narrow the pylorus lumen 40. Ideally, mass(es) of bulking agent is implanted adjacent the intermediate sphincter loop at sites S₁ and S₂ or in various ones of the sites S₁ through S₇ to efficaciously narrow the pylorus lumen 40. Ideally, the mass of bulking agent is implanted in a position that extends across or is closely adjacent the pyloric sphincter 48 so that residual sphincter activity is optimized.

Alternatively, the mass(es) of bulking agent can be implanted in or against the smooth muscle layers of the duodenum 50 to provide bulk to cause the distal and/or intermediate sphincters to contract to obstruct the pylorus lumen 40. The precise number, shape and positioning of the mass(es) of bulking agent depends on the patient's anatomy, and will be a matter of clinical choice at the time of implantation.

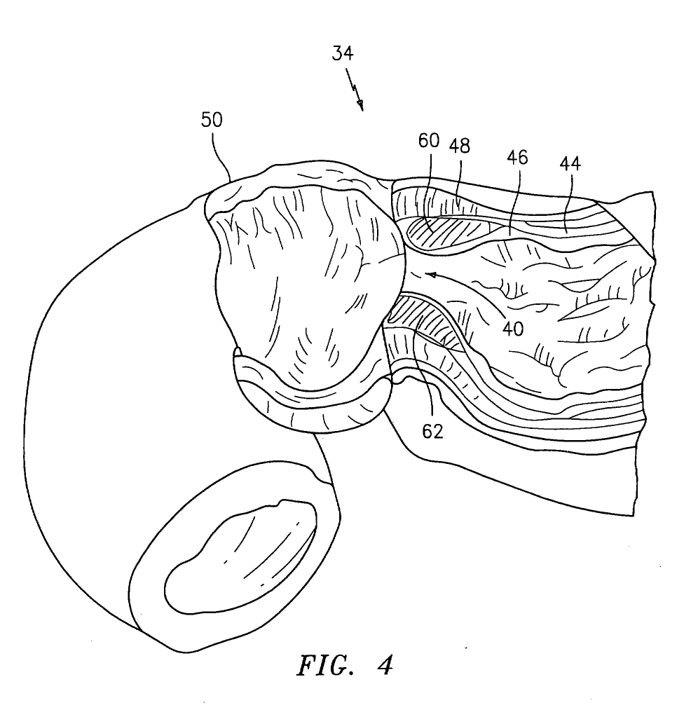

FIG. 4 depicts implanted masses of bulking agent 60 and 62 implanted submucosally adjacent to the pyloric sphincter 48. The particular composition of the masses of bulking agents 60 and 62 can be selected from the compositions in accordance with the disclosure or their equivalents. The particular implantation sites, and the size, shape and number of such implants can be selected by the surgeon to meet the needs of the particular patient.

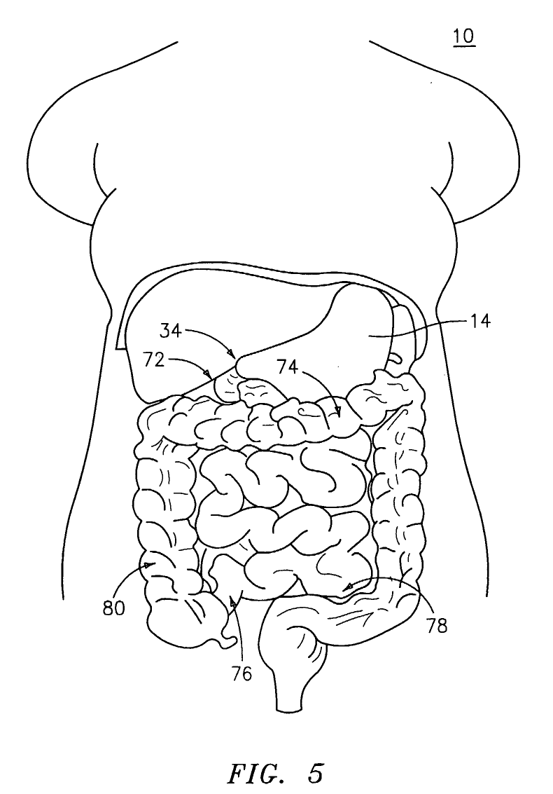

FIG. 5 is a schematic illustration of the GI tract identifying potential implantation sites of one or more mass of bulking agent to restrict a lumen and slow emptying of the contents of the stomach 14, duodenum 50 or small intestines 78. The particular composition of the masses of bulking agent implanted at such sites can be selected from compositions in accordance with the present disclosure or their equivalents. The particular implantation sites, and the size, shape and number of such implants can be selected by the surgeon to meet the needs of the particular patient.

The implantation within the duodenum 50 can be adjacent the first flexure (flexura duodenisuperior) 72 or adjacent the duodenojunal flexure 74. One or more mass of bulking agent can be implanted endoscopically within the wall of the duodenum in a manner similar to the above-described procedure for insertion of the same in relation to the pylorus 34.

One or more mass of bulking agent can be implanted within the wall of the ileocecal sphincter 76 at the junction of base of the ascending colon 80 and the small intestine 78. The ileocecal sphincter 76 opens to allow partially digested chyme to move from the small intestine 78 to the colon 80. Partially constricting the ileocecal sphincter 76 when it is normally relaxed would limit the movement of partially digested food from the small to large intestine, creating a condition similar to pseudo-obstruction (with attendant symptoms of nausea, vomiting, abdominal pain in association with eating). One or more mass of bulking agent can be implanted with the aid of a sigmoidscope or a laparascope within the wall of the ileocecal sphincter 76 in a manner similar to the above-described procedure for insertion of the same in relation to the pylorus 34.

The present compositions can be injected directly into the submucosa to form the mass of bulking agent therein. Alternatively, a space can first be formed in the submucosa by injection of saline solution other aqueous or physiologic solution into the submucosa to form a blister. The amount of the present compositions injected into the submucosal space for each implant can range from 0.01 cc to 10 cc.

If desired, a contrast agent can be incorporated into the present compositions. Such contrast agents comprise biocompatible radiopaque materials that are either water-soluble or water insoluble. Water-soluble contrast agents include metrizamide, iopamidol, iothalamate sodium, iodomide sodium, and meglumine. Well known water insoluble contrast agents include gold, tungsten and platinum powders as well as tantalum powder, tantalum oxide, and barium sulfate, etc. The optional contrast agent in the present compositions permits the mass(es) of bulking agents to be observed entering the site of interest and to be monitored after completion of the procedure so that the stability of the mass and any changes in its shape or location can be observed over time.

### Adhering Grafts

In yet another embodiment, the present disclosure contemplates methods for adhering tissue grafts using the present compositions. Current methods of tissue grafting are complicated by multiple use of sutures, low cosmetic value, wound complications such as foreign body reactions, void and non-adherent grafts. The present methods overcomes problems known in the art. The methods of tissue adhesion described herein are ideal for tissues in need of repair and/or a water-tight seal. These tissues can be of any type where tissue adhesion such as wound closure is necessary, for example a cardiovascular, neurological, gastrointestinal, urological, renal, occular, oral, connective, respiratory, otolaryngological, dermatological, genital, gynecological or musculoskeletal tissue. Wound closure can comprise the joining of cut or otherwise separated edges or surfaces of the damaged tissue. Wound closure can further comprise the grafting of an exogenous tissue on to the surface of a damaged tissue.

The methods described herein are suitable for use in a variety of applications, including in vitro laboratory applications, ex vivo tissue treatments, but especially in in vivo surgical procedures on living subjects, e.g., humans, and non-surgical wound healing.

The methods described herein are particularly useful for surgical applications, e.g., to seal, close, or otherwise join, two or more portions of tissue, e.g., to perform a tissue transplant and/or grafting operation, or to heal damaged tissue, e.g., a corneal incision, or to prevent leakage from tissue. The methods described herein can be used in surgical applications where precise adhesion is necessary, and/or where the application of sutures, staples, or protein sealants is inconvenient or undesirable.

In particularly useful embodiments, the tissue bonding methods described herein can be used in tissue grafting. Exogenous grafts can be, for example, autografts, allografts or xenografts. In one embodiment, an exogenous tissue graft comprising tissue such as skin, muscle, vasculature, stomach, esophagus, colon or intestine, can be placed over the surface of the wound, and contacted with the compositions described herein. The application of the present compositions enables rapid and sustained adherence of the graft to the wound surface and the ability to resist shear stress. Sources of grafted tissue can be any known in the art, including exogenous grafts obtained from non-injured tissues in a subject. Sources of grafted tissue can also comprise extracellular matrix-based scaffolds, such as collagen and proteoglycan, and/or other engineered tissue implants.

Exogenous grafts can likewise be synthetic, e.g. skin substitutes. Synthetic materials suitable for use in grafting include, but are not limited to, silicon, polyurethane, polyvinyl and nylon. Skin substitutes can be any known in the art, including those comprising culture derivatives and cellular or acellular collagen membranes. Culture derived substitutes give rise to bilayer human tissue, for example Apligraf™ comprises epidermal or dermal analogs derived from neonatal foreskin, the host-graft composite of which will become repopulated with cells from the host subject. Commercially available skin substitutes include Biobrane™, composed of silicon, nylon and collagen, TransCyte™, composed of silicon, collagen, fibronectin and glycosaminoglycan, and Integra™, composed of silicon, collagen and glycosaminoglycan. Skin substitutes can be used in applications of permanent and semi-permanent grafting.

In grafting tissues, the surface of the graft is aligned to the lesion site through a process known in the art as “approximation.” Approximation of the graft to the lesion site can be carried out according to methods known in the art. For instance, a graft can be placed on top of the lesion site and aligned so that the dye-stained dermal sides are in close approximation. Molecular contact between the graft and the lesion site is achieved by close approximation, which can be performed through pressing and smoothing the dermal-to-dermal composite with several layers of tissue paper, which are then removed without disturbing the graft interface. The approximated graft-lesion site composite is then ready for application of the present compositions.

### Lumen Occlusion

The present methods also provide for completely or partially blocking, sealing, filling, or adding bulk to various lumens or regions of muscle or tissue within the body of a patient. As used herein, the term “lumen” is intended to encompass the space within various hollow organs or vessels of the body, such as the vas deferens, Fallopian tubes, veins, arteries, intestines, trachea, uterus, and the like. As used herein, the term “closure” is intended to mean the complete or partial blockage, sealing or occlusion, of a space, such as a lumen or channel, which thereby impairs or blocks passage of material through the space.

In an alternative embodiment, the subject methods are useful for a form of birth control or sterility in females, wherein the present compositions are injected, or implanted, such that the Fallopian tubes are filled or blocked thereby preventing egg and/or sperm from passing through or around the biomaterial. Using this approach, pregnancy would be prevented since the ova or eggs located in the Fallopian tubes would not exit to the uterus and would not make contact with sperm. The blockage, and hence the sterility or birth control, is reversible by removal of the present material or re-sectioning of the tube after surgery, wherein the blocked portion of the tube is excised and the remaining portions of the tube are reconnected. It is preferable that the sections of the Fallopian tubes blocked with the present compositions are those directly connected or closest to the uterus. Administration of the present compositions for this therapeutic indication can occur via catheter or via endoscopes, such as a fiberoptic scope, hysteroscope, and the like.

The administration of the present compositions via implant or injection is minimally invasive and usually can be performed on an outpatient basis, resulting in a lower cost than other surgical forms of sterility or birth control. The procedure also eliminates issues of patient compliance, since the patient need not follow any specific instructions or remember to ingest or insert other forms of birth control, such as pills, diaphragms, and the like. However, supplemental forms of birth control can be utilized, if desired, especially those which prevent disease transmission.

According to the most general method of the present disclosure, an effective amount of a composition is administered to the site of a lumen or void within the body of a patient. The term “effective amount”, as used herein, means the quantity of the present composition needed to augment, block, or fill the biological structure of interest. The effective amount of material administered to a particular patient will vary depending upon a number of factors, including: the sex, weight, age, and general health of the patient; the specific type, concentration, and consistency of the material; and the particular site and condition being treated. The material may be administered over a number of treatment sessions.

Applications for the use of the present compositions, methods and kits of the present disclosure include applying the compositions to block or occlude tear ducts, salivary gland ducts, sweat gland ducts, and arteriovenous connections, to treat conditions where such blockage or occlusion is desired. For example, in a condition known as arteriovenous anastomosis, where an artery and a vein are improperly joined, leading to ‘starving’ of cells that are supplied by a capillary bed that is bypassed due to the anastomosis, the improper junction of the anastomosis may be occluded by use of a composition of the present invention, applied to form a blockage of the improper channel, thereby redirecting the arterial flow to the capillary bed.

Another embodiment of the present invention is to cut of the blood supply to a tumor by occluding an artery, and/or a capillary plexus, that directly supplies the tumor.

Another embodiment of the present invention is to form a blockage or occlusion in manmade channels made in bones such as the skull. For instance, a temporary cranial tap may be made by a surgeon to release blood that has pooled between the brain and the skull, such as due to a concussion. A composition of the present invention may be used to fill such a channel after the release of the blood and pressure. This prevents the passage of extra cranial fluids, or pathogens, through the channel.

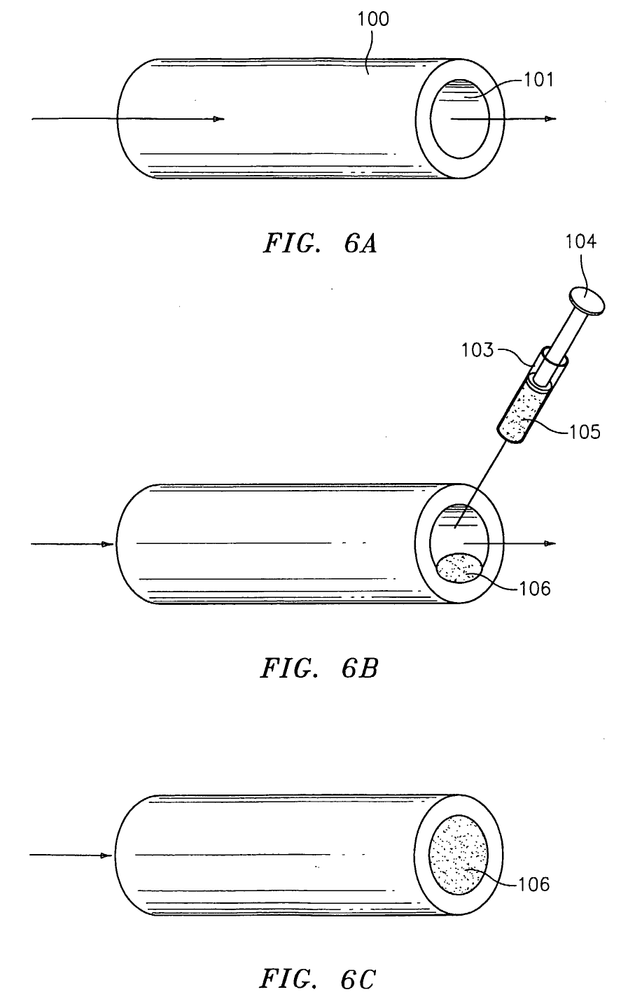

The methods used to form an occlusion or to bulk up tissue are shown in FIGS. 6A-6C. FIG. 6A shows a normal vessel 100 having an unobstructed lumen 101 with a general material flow in the direction of the arrow. FIG. 6B depicts a percutaneous injection via a syringe, 110, having a plunger, 111, and an attached hollow needle 112, and containing a flowable composition, 120 in accordance with the present disclosure. The end of the needle, 112, is positioned in the lumen, 101, of a desired vessel. After confirming proper positioning, as by radiographic or visual means, a desired quantity of the present composition 120 is injected into the lumen 101. Once injected the material forms an occlusion that prevents material movement through that section of the vessel, thereby effectuating sterilization, birth control or other desirable results. See FIG. 6C.

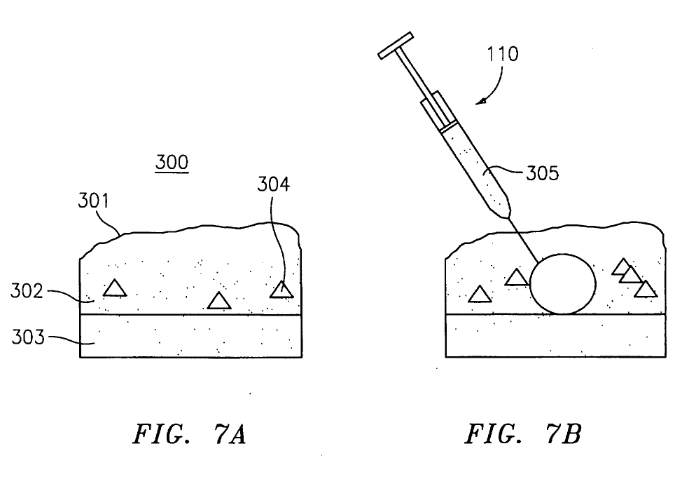

FIGS. 7A-B depict a percutaneous injection of material to bulk up a tissue. FIG. 7A shows a typical skin layer generally indicated at 300 comprising the epidermal layer 301, dermal layer 302 and subdermal layer 303 and associated native cells 304. FIG. 7B shows injection via a syringe 110 of a material 305 to increase the mass of the tissue. An effective amount of material 305 is injected into the dermal layer to cause a desired degree of swelling.

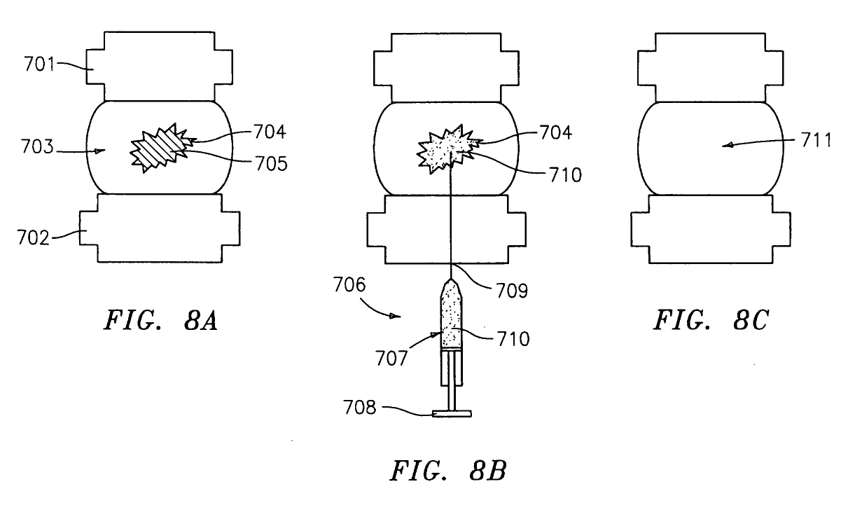

The present compositions may also be applied to a torn annulus fibrosus to cause an adhesion between tissues. The exact combination of substances may vary so long as the composition, once applied is capable of forming a seal through development of tissue adhesion. FIG. 8A shows an intervertebral disc complex generally indicated at 700, comprising an upper vertebrae 701 a lower vertebrae 702 and a ruptured disc 703. As shown, the disc 703 has ruptured along a portion of the annulus fibrosus 704 which has exposed the nucleus pulposus 705. FIG. 8B depicts disc syringe 706 comprising a barrel 707, a plunger 708 and a needle 709. The barrel is filled with a composition 710 in accordance with the disclosure capable of causing an adhesion in tissue of the annulus fibrosus when applied. As shown the composition 710 is injected into the damaged site of the annulus fibrosus 704 to fill in the area. FIG. 8C shows a intervertebral disc complex after application and assimilation of the present composition showing a repaired disc 711.

The compositions, methods and kits of the present invention may be used for the blockage or occlusion of other ducts, channels, and lumens, and the bulking up of tissues and muscles other than those described above, such as may be envisioned and practiced by one of skill in the art.

In the present methods, a sufficient quantity of material may be injected as a bolus, expanding the lumen and effectively closing it off. Depending on the site of injection, this method may result in the narrowing or the closure of a body opening. For instance, where it is medically desirable or necessary to close the cervical opening of the uterus, an injection of the preparation at or near the cervical opening results in adhesion formation (also known as scar tissue or granulation tissue formation) that closes the uterus. More broadly, this method can be applied to a wider range of medical conditions where it is desirable to close or narrow an opening.

### Decortication

In another embodiment the present disclosure contemplates the use of the present compositions to treat tissue after a surgical procedure involving decortication. In one such embodiment, after lung tissue is separated from the wall of the chest cavity, the present compositions are applied to the chest cavity wall, the outer surface of the lung, or both to assist in healing. Other surgical procedures involving decortication in which application of the present composition will assist in healing will be apparent to those skilled in the art.

As used herein the terms “administered”, “implanted”, or “implantation” are used interchangeably and mean that the material is delivered to the area of treatment by techniques know to those skilled in the art and appropriate for the disease to be treated. Both invasive and non-invasive methods may be used for delivery. “Injectable” as used in the present disclosure means capable of being administered, delivered or carried into the body via needle or other similar ways.

“Skin wrinkles,” “skin deficiencies,” and “skin contour deficiencies” are used interchangeable in the present disclosure to refer to skin conditions that are either abnormal or undesirable due to various internal or external conditions such as aging, environmental exposure to the sun and other elements, weight loss, child bearing, disease such as acne and cancer, surgery, wounds, accidents, bites, cuts. “Dermal augmentation” in the context of the present disclosure refers to any change of the natural state of a mammal's skin and related areas due to external acts. The areas that may be changed by dermal augmentation include, but not limited to, epidermis, dermis, subcutaneous layer, fat, arrector pill muscle, hair shaft, sweat pore, and sebaceous gland. “Tissue bulking” in the context of the present disclosure refers to any change of the natural state of a mammal's non-dermal soft tissues due to external acts or effects. The tissues encompassed by the disclosure include, but not limited to, muscle tissues, connective tissues, fats, and, nerve tissues. The tissues encompassed by the present disclosure may be part of many organs or body parts including, but not limited to, the sphincter, the bladder sphincter and urethra.

An effective amount of one or more biologically active agent, such as a wound healing agent, antibiotic, or antimicrobial agent, can be incorporated into the present compositions. In this context, an “effective amount” refers to the amount of biologically active agent, antibiotic, or antimicrobial agent required to obtain the desired therapeutic effect, such as improved or accelerated healing of the defect or void, or prevention of infection at the site of administration.

“Biologically active agent” as used herein includes, but is not limited to, antiviricides, particularly those effective against viruses such as HIV and hepatitis; nonoxynol-9; chlorhexidine; benzalkonium chloride; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin, etc.; amino acids, magainins, peptides, vitamins, inorganic elements, co-factors for protein synthesis; hormones; endocrine tissue or tissue fragments; enzymes such as collagenase, peptidases, oxidases, etc.; polymer cell scaffolds with parenchymal or other cells; surface cell antigen eliminators; angiogenic or angiostatic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, growth factors, growth hormones such as somatotropin; bone digestors; antitumor agents; glycosaminoglycans, proteoglycans, fibronectin; cellular attractants and attachment agents; immuno-suppressants; adjuvants such as Freunds complete adjuvant, lipopoylcaccharides, vegetable oil, etc.; permeation enhancers, e.g., fatty acid esters such as laureate, myristate and stearate monoesters of polyethylene glycol, enamine derivatives, alpha-keto aldehydes, etc.; nucleic acids; bioerodable polymers such as those disclosed in U.S. Pat. Nos. 4,764,364 and 4,765,973, and combinations of any of the foregoing. The amounts of such medically useful substances can vary widely with optimum levels being readily determined in a specific case by routine experimentation.

The term “growth factor” as used herein refers to a polynucleotide molecule, polypeptide molecule, or other related chemical agent that is capable of effectuating differentiation or proliferation of cells. Examples of growth factors as contemplated for use in accord with the teachings herein include a epidermal growth factor (EGF), transforming growth factor-alpha (TGF-alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), and/or platelet derived growth factor (PDGF).

As used herein, the term “effective amount”, in reference to a biologically active agent, also refers to that amount of material which is pharmaceutically and physiologically acceptable to the particular patient undergoing treatment. In some embodiments, the composition is functionalized by chemical coupling with a marker, which can be:
a chemical dye, such as Cibacron Blue or Procion Red HE-3B, making possible a direct visualization of the compositions (Boschetti, J. Biochem—Biophys. Meth., 19:21-36 (1989)). Examples of functionalized monomer usable for this type of marking N-acryloyl hexamethylene Cibacrone Blue or N-acryloyl hexamethylene Procion Red HE-3B;
a magnetic resonance imaging agent (erbium, gadolinium or magnetite);
a contrasting agent, such as barium or iodine salts, (including for example acylamino-e-propion-amido)-3-triiodo-2,4,6-benzoic acid, which can be prepared under the conditions described by Boschetti et al. (Bull. Soc. Chim., No. 4 France, (1986)). In the case of barium or magnetite salts, they can be directly introduced in powered form in the initial composition.

As indicated above it is also possible to mark the compositions after their synthesis. This can be done, for example, by grafting of fluorescent markers derivatives (including for example fluorescein isothiocyanate (FITC), rhodamine isothiocyanate (RITC) and the like).

The compositions of the present disclosure also can be chemically modified so that they will “carry” therapeutic effects, vascularization effects, anti-vascularization effects, visualization properties, anti-inflammatory effects, anti-bacterial effects, or anti-histamine effects, or combinations thereof. The chemical modification of the compositions of the present disclosure is made possible by the fact that the compositions comprise particles made of polymers that are crosslinked so that they can contain chemicals within their structures that possess various properties and that they possess unique characteristics associated with surface covalent bonds. The chemical modification of the compositions of the present disclosure may also occur through the interactions between the compositions and the neighboring cells and tissue after the administration.

The present disclosure provides a method for causing tissue bulking in a mammal. The method comprises administering a composition in accordance with this disclosure to the mammal. The composition is injectable through a needle (e.g., of about 18 to about 26 gauge) and the compositions are not capable of being digested or eliminated by macrophage or other elements of said mammal's lymphatic system. The tissue bulking method of the present disclosure is suitable for the treatment of various tissue defects including, but not limited to, dental tissue defects, vocal cord tissue defects, or other non-dermal soft tissue defects.

The injection method of the present disclosure can be carried out by any type of sterile needles and corresponding syringes or other means for injection, such as a three-way syringe. The injection is preferably made into the area that needs tissue bulking treatment. The needles, syringes and other means for injection are commercially available from various suppliers such as VWR Scientific Products (West Chester, Pa.), Becton Dickinson, Kendal, and Baxter Healthcare. The size of the syringe and the length of the needle used will dependent on the particular injection based on factors such as the specific disease or disorders being treated, the location and depth of the injection, and the volume and specific composition of the injectable suspension being used. A skilled practitioner will be able to make the selection of syringe and needle based on experience and the teaching of the present disclosure.

The present disclosure additionally provides a kit for performing bulking, dermal augmentation tissue bulking, tissue bulking and/or occlusion. The kit comprises a needle and a corresponding syringe (both of which are sterile), wherein the syringe optionally contains a composition in accordance with the present disclosure. The composition is injectable through the needle and the compositions are not capable of being eliminated by macrophage or other elements of said mammal's immune or lymphatic system. Alternatively, the kit comprises a needle, a corresponding syringe, and separate containers containing the compositions in dried and sterilized form and a biocompatible solvent. The dried sterilized compositions and the solvent are ready to be mixed for injection either in their respective containers or in the syringe. These kits are sterile and ready to use. The kits are designed in various forms based the sizes of the syringe and the needles and the volume of the injectable composition contained therein, which in turn are based on the specific skin or tissue defects the kits are designed to treat.

The embodiments of the present disclosure described above are intended to be merely exemplary and those skilled in the art will recognize, or be able to ascertain using no more than routine experimentation, numerous equivalents to the specific procedures described herein. All such equivalents are considered to be within the scope of the present disclosure and are covered by the following claims.

The contents of all references described herein are hereby incorporated by reference.

1. A method comprising injecting a composition containing a biocompatible, non-bioabsorbable isocyanate-based material, into a tissue selected from the group consisting of anal sphincter, bladder sphincter and ileocecal sphincter in an amount sufficient for bulking the tissue.

2. A method as in claim 1 wherein the composition further comprises an imaging agent.

3. A method as in claim 1 wherein the composition further comprises an aniti-inflammatory agent.

4. A method as in claim 1 wherein the composition further comprises a bioactive agent.

5. A method as in claim 1 wherein the amount of composition injected is from about 0.01 cc to about 10 cc.

6. A method comprising injecting a composition containing a biocompatible, non-bioabsorbable isocyanate-based material into tissue in an amount sufficient to augment the tissue.

7. A method as in claim 6 wherein the composition is injected subcutaneously.

8. A method as in claim 6 wherein the composition is injected to enhance the size or appearance of a subject's cheeks, lips, breast or penis.

9. A method as in claim 6 wherein the composition further comprises an imaging agent.

10. A method as in claim 6 wherein the composition further comprises an aniti-inflammatory agent.

11. A method as in claim 6 wherein the composition further comprises a bioactive agent.

12. A method as in claim 6 wherein the amount of composition injected is from about 0.01 cc to about 10 cc.

13. A method comprising securing a graft to a site of tissue defect using a composition containing a biocompatible, non-bioabsorbable isocyanate-based material in an amount sufficient to secure the graft to the tissue.

14. A method as in claim 13 wherein the composition further comprises an imaging agent.

15. A method as in claim 13 wherein the composition further comprises an aniti-inflammatory agent.

16. A method as in claim 13 wherein the composition further comprises a bioactive agent.

17. A method as in claim 13 wherein the amount of composition injected is from about 0.01 cc to about 10 cc.

18. A method as in claim 13 wherein the graft is an exogenous graft.

19. A method as in claim 13 wherein the graft is a skin substitute.

20. A method comprising administering a composition containing a biocompatible, non-bioabsorbable isocyanate-based material into a lumen in an amount sufficient to achieve closure of the lumen.

21. A method as in claim 20 wherein the composition further comprises an imaging agent.

22. A method as in claim 20 wherein the composition further comprises an aniti-inflammatory agent.

23. A method as in claim 20 wherein the composition further comprises a bioactive agent.

24. A method as in claim 20 wherein the lumen is a Fallopian tube.

25. A method comprising administering a composition containing a biocompatible, non-bioabsorbable isocyanate-based material into a manmade channel in an amount sufficient to achieve closure of the manmade channel.

26. A method as in claim 25 wherein the manmade channel is a cranial tap.

27. A method comprising administering a composition containing a biocompatible, non-bioabsorbable isocyanate-based material between tissue separated during a surgical procedure involving decortication.

28. A kit for bulking, augmenting or occluding tissue comprising at least one sterile needle, at least one sterile syringe, and a composition containing a biocompatible, non-bioabsorbable isocyanate-based material.

29. The kit of claim 28 further comprising a biocompatible solvent.

30. The kit of claim 28 wherein the at least one sterile syringe comprises an about 18 to about 26 gauge needle.

31. The kit of claim 28 wherein the at least one syringe is a three-way syringe.
